(19) United States
(12) Patent Application Publication
(10) Pub. No.: US 2023/0346384 A1
(43) Pub. Date: Nov. 2, 2023

(54) LEFT ATRIAL APPENDAGE CLOSURE DEVICE WITH CENTRAL SUPPORT

(71) Applicant: BOSTON SCIENTIFIC SCIMED, INC., Maple Grove, MN (US)

(72) Inventors: JOSEPH WALKER, Shoreview, MN (US); ROHITH JAYARAM, Maple Grove, MN (US)

(73) Assignee: BOSTON SCIENTIFIC SCIMED, INC., Maple Grove, MN (US)

(21) Appl. No.: 18/139,543

(22) Filed: Apr. 26, 2023

Related U.S. Application Data
(60) Provisional application No. 63/335,571, filed on Apr. 27, 2022.

(51) Int. Cl. A61B 17/12

(52) U.S. Cl. CPC A61B 17/12122; A61B 17/12031; A61B 17/12172; A61B 2017/00632

(57) ABSTRACT

An implantable medical device includes an expandable frame moveable between a collapsed configuration for delivery, an expanded configuration for deployment, and a distended configuration. The expandable frame includes a first frame apex and a second frame apex, a distance between the first frame apex and the second frame apex while within the expanded configuration defining an expanded configuration apex distance, a distance between the first frame apex and the second frame apex while in the distended configuration defining a distended configuration apex distance. A support member extends between the first frame apex and the second frame apex, the support member including a first end secured to the first frame apex and a second end releasably securable relative to the second frame apex, the adjustable support adapted to limit the distended configuration apex distance relative to the expanded configuration apex distance.

### CROSS-REFERENCE TO RELATED APPLICATIONS

This application claims the benefit of priority of U.S. Provisional Application No. 63/335,571 filed Apr. 27, 2022, the entire disclosure of which is hereby incorporated by reference.

### TECHNICAL FIELD

The present disclosure pertains to medical devices, and methods for manufacturing and using medical devices. More particularly, the disclosure is directed to implantable medical devices having a central support limiting axial expansion.

### BACKGROUND

A wide variety of medical devices have been developed for medical use, for example, for use in accessing body cavities and interacting with fluids and structures in body cavities. Some of these devices may include guidewires, catheters, pumps, motors, controllers, filters, grinders, needles, valves, and delivery devices and/or systems used for delivering such devices. These devices are manufactured by any one of a variety of different manufacturing methods and may be used according to any one of a variety of methods. Of the known medical devices and methods, each has certain advantages and disadvantages.

### SUMMARY

This disclosure provides design, material, manufacturing method, and use alternatives for medical devices. As an example, an implantable medical device includes an expandable frame defining a body of the implantable medical device, the expandable frame moveable between a collapsed configuration for delivery and an expanded configuration for deployment, the expandable frame including a first frame apex and a second frame apex, the expandable frame axially extendable beyond the expanded configuration in response to an applied axial force. An adjustable support extends between the first frame apex and the second frame apex and is adapted to limit how far the expandable frame is able to extend axially beyond its expanded configuration.

Alternatively or additionally, the adjustable support may include a first end secured to the first frame apex and a second end releasably securable to the second frame apex.

Alternatively or additionally, the adjustable support may include a member having a first end that is secured to the first frame apex.

Alternatively or additionally, the member may include a second end that is releasably securable relative to the second frame apex.

Alternatively or additionally, the adjustable support may further include a pin secured to the second frame apex, with the member extending through the pin.

Alternatively or additionally, the adjustable support may further include a tapered collar secured in position on the member, the tapered collar adapted to be releasably secured to the pin in order to secure the second end of the member relative to the second frame apex.

Alternatively or additionally, the member may include a suture that is wrapped over the first frame apex and has a first free end and a second free end that each extend proximally beyond the second frame apex.

Alternatively or additionally, the adjustable support may further include a ratcheting element, the ratcheting element including a toothed lumen through which the first free end and the second free end of the suture extends, the toothed lumen adapted to allow the ratcheting element to slide in one direction relative to the suture while restricting movement in an opposing direction.

Alternatively or additionally, the adjustable support may further include a reversible suture lock that includes a suture lock body defining a lumen extending therethrough, a first element slidingly disposed within the lumen, the first element including a first element lumen through which the member is free to extend, a second element slidingly disposed within the lumen and engaged with the first element, the element member including a second element lumen through which the member is free to extend, and an engagement element that is adapted to releasably engage the first element, the engagement element including an engagement element lumen through which the member is free to extend. The first element and the second element are moveable between a first position in which the member is free to move relative to the suture lock body and a second position in which the member is constrained against movement relative to the suture lock body.

Alternatively or additionally, the adjustable support may further include a suture lock including a suture lock body defining a lumen extending therethrough, the lumen accommodating the member therethrough, and a ratchet that engages the member, wherein the ratchet holds a tensile force on the member.

Alternatively or additionally, the implantable medical device includes an LAAC (left atrial appendage closure) device.

Alternatively or additionally, the expandable frame may be biased to the expanded configuration.

As another example, an implantable medical device includes an expandable frame defining a body of the implantable medical device, the expandable frame moveable between a collapsed configuration for delivery, an expanded configuration for deployment, and a distended configuration. The expandable frame includes a first frame apex and a second frame apex, a distance between the first frame apex and the second frame apex while within the expanded configuration defining an expanded configuration apex distance, a distance between the first frame apex and the second frame apex while in the distended configuration defining a distended configuration apex distance. A support member extends between the first frame apex and the second frame apex, the support member including a first end secured to the first frame apex and a second end releasably securable relative to the second frame apex, the adjustable support adapted to limit the distended configuration apex distance relative to the expanded configuration apex distance.

Alternatively or additionally, the implantable medical device may further include a pin secured to the second frame apex, with the support member extending through the pin, and a tapered collar secured in position on the support member, the tapered collar adapted to be releasably secured to the pin in order to secure the second end of the support member relative to the second frame apex.

Alternatively or additionally, the support member may include a suture that is wrapped over the first frame apex and has a first free end and a second free end that each extend proximally beyond the second frame apex.

Alternatively or additionally, the implantable medical device may further include a ratcheting element, the ratcheting element including a toothed lumen through which the first free end and the second free end of the suture extends, the toothed lumen adapted to allow the ratcheting element to slide in one direction relative to the suture while restricting movement in an opposing direction.

Alternatively or additionally, the implantable medical device may further include a reversible suture lock that includes a suture lock body defining a lumen extending therethrough, a first element slidingly disposed within the lumen, the first element including a first element lumen through which the suture is free to extend, a second element slidingly disposed within the lumen and engaged with the first element, the element member including a second element lumen through which the suture is free to extend, and an engagement element that is adapted to releasably engage the first element, the engagement element including an engagement element lumen through which the suture is free to extend. The first element and the second element are moveable between a first position in which the suture is free to move relative to the suture lock body and a second position in which the suture is constrained against movement relative to the suture lock body.

Alternatively or additionally, the implantable medical device may further include a suture lock including a suture lock body defining a lumen extending therethrough, the lumen accommodating the suture therethrough, and a ratchet that engages the suture, where the ratchet maintains a tensile force on the suture.

As another example, an LAAC (left atrial appendage closure) device includes an expandable frame defining a body of the implantable medical device, the expandable frame moveable between a collapsed configuration for delivery, an expanded configuration for deployment, and a distended configuration. The expandable frame includes a first frame apex and a second frame apex, a distance between the first frame apex and the second frame apex while within the expanded configuration defining an expanded configuration apex distance, a distance between the first frame apex and the second frame apex while in the distended configuration defining a distended configuration apex distance. A support member extends between the first frame apex and the second frame apex, the support member including a first end secured to the first frame apex and a second end releasably securable relative to the second frame apex. An adjustable support is adapted to limit the distended configuration apex distance relative to the expanded configuration apex distance.

Alternatively or additionally, the adjustable support may further include a pin secured to the second frame apex, with the member extending through the pin, and a tapered collar secured in position on the member, the tapered collar adapted to be releasably secured to the pin in order to secure the second end of the member relative to the second frame apex.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a side view of an illustrative implantable medical device including an illustrative adjustable support;

FIG. 2A is a schematic illustration of relative sealing for an illustrative implantable medical device without an adjustable support;

FIG. 2B is a schematic illustration of relative sealing for the illustrative implantable medical device of FIG. 1;

FIG. 3A is a side view of an illustrative implantable medical device lacking an adjustable support, shown in a distended configuration;

FIG. 3B is a side view of the illustrative implantable medical device of FIG. 1, shown in a distended configuration;

FIG. 4A is a side view of an illustrative implantable medical device including an illustrative adjustable support;

FIG. 4B is an enlarged side view of a portion of the illustrative adjustable support of FIG. 4A;

FIG. 5A is a cross-sectional view of an illustrative suture locking device that may be used with the illustrative implantable medical device of FIG. 1, shown in an unlocked configuration in which a suture is not constrained against axial movement;

FIG. 5B is a cross-sectional view of the illustrative suture locking device of FIG. 5A, shown in a locked configuration in which a suture is constrained against axial movement; and

FIG. 6 is a cross-sectional view of an illustrative suture locking device that may be used with the illustrative implantable medical device of FIG. 1.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

A variety of medical devices are intended to be implanted within a patient. In some cases, once an implantable medical device has been implanted, or has undergone at least the initial stages of being implanted, the implantable medical device may undergo a tug test prior to implantation being completed. It will be appreciated that in some cases, the tug test may cause the implantable medical device to become distended. In some cases, this can cause the implantable medical device to become dislodged. In cases of implantable medical devices that are intended to seal against tissue, for example, the distension that occurs during testing such as a tug test may cause a loss of sealing against the tissue.

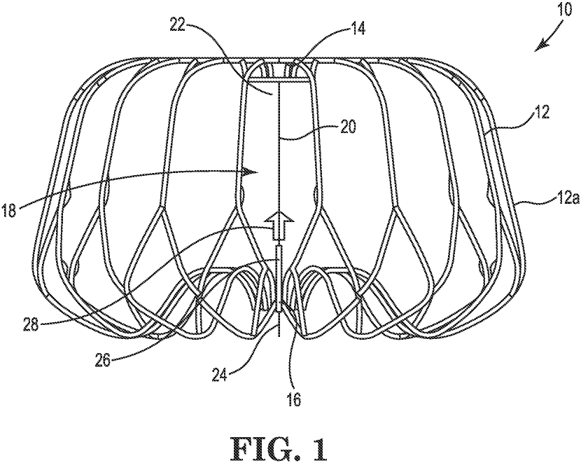

FIG. 1 is a side view of an illustrative IMD (implantable medical device) 10. The illustrative IMD 10 may be adapted to be used as an LAAC (left atrial appendage closure) device. An LAAC is used to seal off an interior of a left atrial appendage from the rest of the patient’s heart in order to reduce or eliminate the possibility of blood clots being dislodged from the left atrial appendage.

The IMD 10 includes an expandable frame 12 that is moveable between a collapsed configuration for delivery and an expanded configuration (as shown in FIG. 1) for deployment. The expandable frame 12 may be configured to reach a distended configuration in response to a tensile force being applied to the expandable frame 12. In some instances, the expandable frame 12 may be biased to the expanded configuration.

In some cases, the IMD 10 may include a membrane (not shown) covering at least a portion of the expandable frame 12 so that by virtue of including the membrane, the IMD 10 is able to seal off the left atrial appendage. The expandable frame 12 is formed of a number of individual frame struts 12a. In some cases, the individual frame struts 12a may be laser cut, for example. Further details regarding the construction of the expandable frame 12 may be found in U.S. Pat. Application Publication No. 2017/0224354, which reference is incorporated by reference herein.

The IMD 10 includes a first frame apex 14 and a second frame apex 16. In some instances, the first frame apex 14 may be considered as being a distal frame apex and the second frame apex 16 may be considered as being a proximal frame apex 16, with distal and proximal being relative terms referring to the eventual implantation orientation of the IMD 10. In some instances, if the IMD 10 includes a membrane, the membrane may extend over the second frame apex 16. In some cases, the second frame apex 16 may be configured to allow attachment of the IMD 10 to a delivery device for delivering the IMD 10 to an appropriate implantation site within a patient, such as within the patient’s heart proximate the left atrial appendage.

The IMD 10 includes an adjustable support structure 18 that may be used to limit distension of the expandable frame 12 during tug testing, for example. The adjustable support structure 18 may be adapted to limit how far the expandable frame 12 is able to extend axially beyond its expanded configuration, into a distended configuration. In some cases, a distance between the first frame apex 14 and the second frame apex 16 while the expandable frame 12 is in the expanded configuration defines an expanded configuration apex distance. In some cases, a distance between the first frame apex 14 and the second frame apex 16 while the expandable frame 12 is in the distended configuration defines a distended configuration apex distance. The adjustable support 18 may be adapted to limit the distended configuration apex distance relative to the expanded configuration apex distance. The expanded configuration apex distance may be in a range of 15 to 22 millimeters (mm), for a 20 mm device and a 40 mm device, respectively. In some cases, the distended configuration apex distance may be in a range of 24 to 44 mm, for a 20 mm device and a 40 mm device, respectively.

In some cases, the adjustable support structure 18 includes an elongate member 20. The elongate member 20 may be flexible or rigid, for example, and may be made of any suitable material. If the elongate member 20 is rigid, the elongate member 20 may be metallic or a rigid polymer. If the elongate member 20 is flexible, the elongate member 20 may be formed of a softer polymer. In some cases, the elongate member 20 may be a thread or a suture. In some cases, the elongate member 20 may be a suture that is wrapped over the first frame apex 14, with a pair of free ends that each extend proximally beyond the second frame apex 16.

The elongate member 20 has a first end 22 that may be secured to the first frame apex 14. The first end 22 may be tied to the first frame apex 14, for example, or may be wrapped around the first frame apex 14. In some cases, the first end 22 may be adhesively secured to the first frame apex 14. If the elongate member 20 is metallic, the first end 22 of the elongate member 22 may be welded or soldered in place to the first frame apex 14. The first end 22 may be secured to the first frame apex 14 without interfering with an ability of the expandable frame 12 to move between its collapsed configuration for delivery and its expanded configuration for deployment.

The elongate member 20 extends through an interior of the expandable frame 12 to a second end 24 that, as can be seen, extends beyond the second frame apex 16. In some cases, the adjustable support structure 18 includes a pin 26 that may be secured to the second frame apex 16, with the elongate member 20 extending through a lumen formed within the pin 26. In some instances, the pin 26 may be radiopaque, in order to enhance visibility during imaging procedures.

In some instances, the adjustable support structure 18 includes a tapered collar 28 that may be secured to the elongate member 20. The tapered collar 28 may be configured to attach to the pin 26 in order to secure the elongate member 20 relative to the pin 26, and thus relative to the second frame apex 16. In some cases, the tapered collar 28 may itself be radiopaque, in addition to or instead of the pin 26 being radiopaque. It will be appreciated that the relative position of the collar 28 on the elongate member 20 will determine whether and to what extent the expandable frame 12 may be distended when the second frame apex 16 is subjected to a tensile force during a tug test, for example.

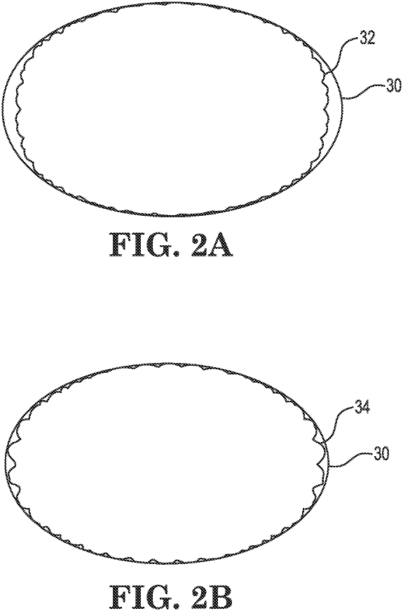

FIGS. 2A and 2B provide a graphical comparison between the sealing ability of an IMD 10 lacking an adjustable support structure such as the adjustable support structure 18 (FIG. 2A) during a tug test and the sealing ability of the IMD 10 with the adjustable support structure 18 (FIG. 2B) during a tug test. In FIG. 2A, an outer circle 30 schematically represents a periphery of tissue against which the IMD 10 is being implanted. An inner circle 32 schematically shows a periphery of the IMD 10 lacking central support. In this example, 94.9 percent of the outer circle 30 is filled by the IMD 10. In FIG. 2B, an inner circle 34 schematically shows a periphery of the IMD 10 that includes central support. In this example, 97.3 percent of the outer circle 30 is filled by the IMD 10.

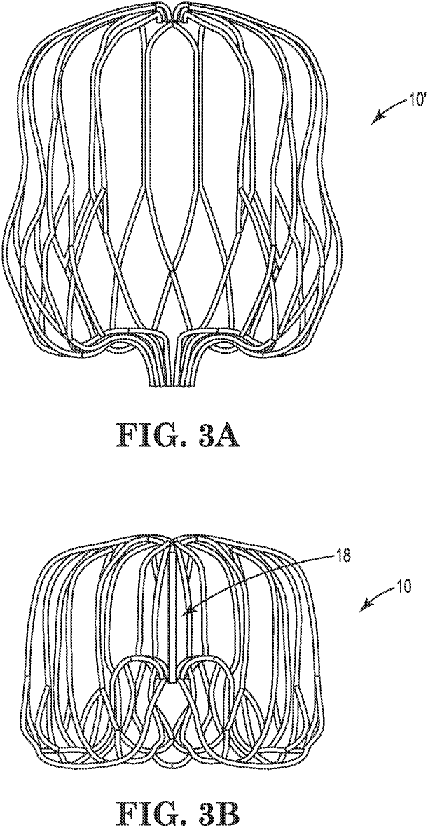

FIG. 3A shows an IMD 10′ during a tug test. The IMD 10′ lacks central support, and as can be seen, has been significantly distended by the tensile forces applied during a tug test. It will be appreciated that distending the IMD 10′ in the axial direction, as shown, causes the IMD 10′ to reduce in diameter. In comparison, FIG. 3B shows the IMD 10 during a tug test. By virtue of including the adjustable support structure 18, and in comparing with FIG. 3A, it can be seen that the adjustable support structure 18 significantly reduces the level of distension of the IMD 10.

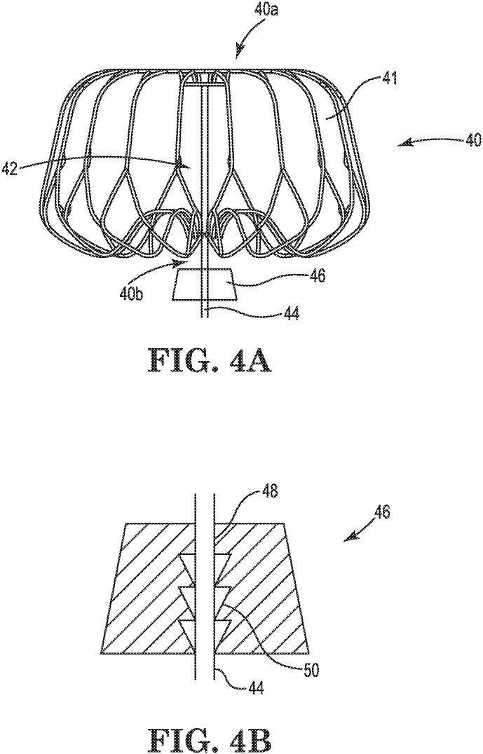

FIG. 4A is a schematic view of an illustrative IMD 40 that may be considered as being an example of the IMD 10. The IMD 40 includes an expandable frame 41 that is moveable between a collapsed configuration for delivery and an expanded (as shown) configuration for deployment. The expandable frame 41 extends between a first frame apex 40a and a second frame apex 40b. In some cases, the first frame apex 40a may be considered as being a distal frame apex and the second frame apex 40b may be considered as being a proximal frame apex.

The IMD 40 includes an adjustable support structure 42 that may be used with the IMD 10, for example. The adjustable support structure 42 includes an elongate member 44 that extends through the IMD 40, and may be attached to the first frame apex 40a and extends downwardly (in the illustrated orientation) and through the second frame apex 40b. In some cases, the elongate member 44 may be a suture, for example. The elongate member 44 extends through a ratchet element 46, better seen in FIG. 4B.

The ratchet element 46 includes a ratchet lumen 48 extending axially through the ratchet element 46. The ratchet lumen 48 includes a number of ratchet teeth 50 that are dimensioned to engage the elongate member 44. In some cases, the ratchet lumen 48 and the ratchet teeth 50 are dimensioned to allow the elongate member 44 to be pulled down through the ratchet element 46, thereby tightening the elongate member 44, but to not allow the elongate member 44 to move in an opposing direction that would loosen the elongate member 44.

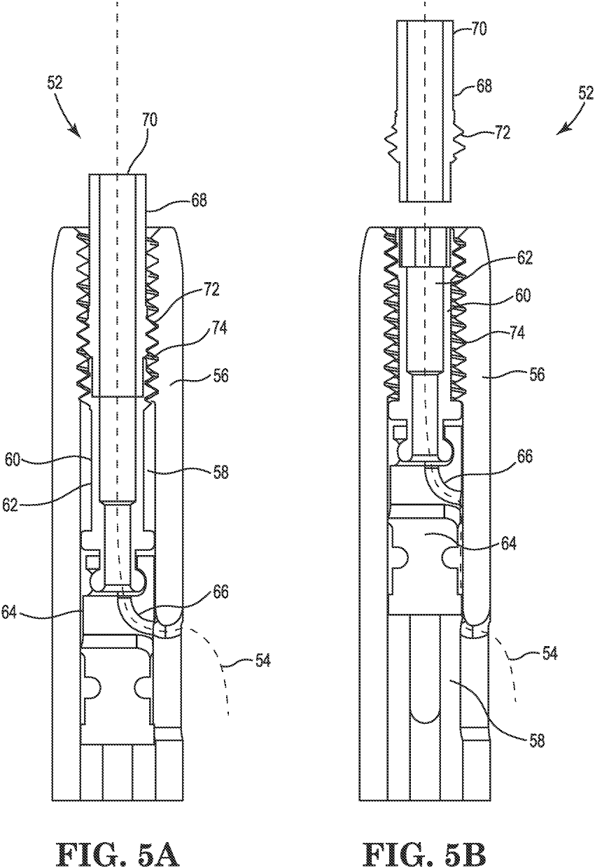

FIGS. 5A and 5B are cross-sectional view of an illustrative suture locking device 52 that may be used with the illustrative IMD 10 of FIG. 1. FIG. 5A shows an unlocked configuration in which a suture 54 is not constrained against axial movement while FIG. 5B shows a locked configuration in which the suture 54 is constrained against axial movement. The suture locking device 52 includes a body 56 defining a body lumen 58 extending through the body 56. The suture locking device 52 includes a first element 60 that is slidingly disposed within the body lumen 58. The first element 56 including a first element lumen 62 through which the suture 54 is free to extend.

The suture locking device 52 includes a second element 64 that is slidingly disposed within the body lumen 58. The second element 64 is engaged with the first element 60 and includes a second element lumen 66 through which the suture 54 is free to extend. The suture locking device 52 includes an engagement element 68 that is adapted to releasably engage the first element 60. The engagement element 68 includes an engagement element lumen 70 through which the suture 54 is free to extend. In some cases, the engagement element 68 includes teeth 72 that interact with a toothed surface 74 formed within the suture lock body 56

The first element 60 and the second element 64 are moveable between a first position (shown in FIG. 5A) in which the suture 54 is free to move relative to the suture lock body 56 and a second position (shown in FIG. 5B) in which the suture 54 is constrained against movement relative to the suture lock body 56. As shown in FIG. 5B, the suture 54 is trapped between the second element 64 and the side wall of the body lumen 58.

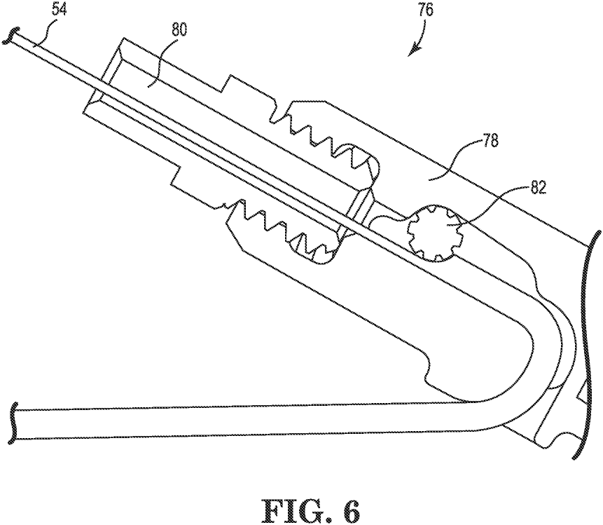

FIG. 6 is a cross-sectional view of an illustrative suture locking device 76 that may be used with the illustrative IMD 10 of FIG. 1. The suture locking device 76 includes a suture lock body 78 defining a lumen 80 extending therethrough. The lumen 80 is adapted to accommodate the suture 54 extending through the lumen 80. A ratchet 82 is adapted to engage the suture 54. Rotating the ratchet 82 will allow slack to be removed from the suture 54.

The devices described herein, as well as various components thereof, may be manufactured according to essentially any suitable manufacturing technique including molding, casting, mechanical working, and the like, or any other suitable technique. Furthermore, the various structures may include materials commonly associated with medical devices such as metals, metal alloys, polymers, metal-polymer composites, ceramics, combinations thereof, and the like, or any other suitable material. These materials may include transparent or translucent materials to aid in visualization during the procedure. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; combinations thereof, and the like; or any other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane, polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), polycarbonates, ionomers, biocompatible polymers, other suitable materials, or mixtures, combinations, copolymers thereof, polymer/metal composites, and the like.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention’s scope is, of course, defined in the language in which the appended claims are expressed.

What is claimed is:

1. An implantable medical device, comprising:
an expandable frame defining a body of the implantable medical device, the expandable frame moveable between a collapsed configuration for delivery and an expanded configuration for deployment, the expandable frame including a first frame apex and a second frame apex, the expandable frame axially extendable beyond the expanded configuration in response to an applied axial force; and
an adjustable support extending between the first frame apex and the second frame apex, the adjustable support adapted to limit how far the expandable frame is able to extend axially beyond its expanded configuration.

2. The implantable medical device of claim 1, wherein the adjustable support comprises a first end secured to the first frame apex and a second end releasably securable to the second frame apex.

3. The implantable medical device of claim 1, wherein the adjustable support comprises a member having a first end that is secured to the first frame apex.

4. The implantable medical device of claim 1, wherein the member comprises a second end that is releasably securable relative to the second frame apex.

5. The implantable medical device of claim 4, wherein the adjustable support further comprises a pin secured to the second frame apex, with the member extending through the pin.

6. The implantable medical device of claim 4, wherein the adjustable support further comprises a tapered collar secured in position on the member, the tapered collar adapted to be releasably secured to the pin in order to secure the second end of the member relative to the second frame apex.

7. The implantable medical device of claim 4, wherein the member comprises a suture that is wrapped over the first frame apex and has a first free end and a second free end that each extend proximally beyond the second frame apex.

8. The implantable medical device of claim 7, wherein the adjustable support further comprises a ratcheting element, the ratcheting element including a toothed lumen through which the first free end and the second free end of the suture extends, the toothed lumen adapted to allow the ratcheting element to slide in one direction relative to the suture while restricting movement in an opposing direction.

9. The implantable medical device of claim 4, wherein the adjustable support further comprises a reversible suture lock comprising:
a suture lock body defining a lumen extending therethrough;
a first element slidingly disposed within the lumen, the first element including a first element lumen through which the member is free to extend;
a second element slidingly disposed within the lumen and engaged with the first element, the element member including a second element lumen through which the member is free to extend;
an engagement element that is adapted to releasably engage the first element, the engagement element including an engagement element lumen through which the member is free to extend;
wherein the first element and the second element are moveable between a first position in which the member is free to move relative to the suture lock body and a second position in which the member is constrained against movement relative to the suture lock body.

10. The implantable medical device of claim 4, wherein the adjustable support further comprises a suture lock comprising:
a suture lock body defining a lumen extending therethrough, the lumen accommodating the member therethrough; and
a ratchet that engages the member;
wherein the ratchet holds a tensile force on the member.

11. The implantable medical device of claim 1, comprising an LAAC (left atrial appendage closure) device.

12. The implantable medical device of claim 1, wherein the expandable frame is biased to the expanded configuration.

13. An implantable medical device, comprising:
an expandable frame defining a body of the implantable medical device, the expandable frame moveable between a collapsed configuration for delivery, an expanded configuration for deployment, and a distended configuration;
the expandable frame including a first frame apex and a second frame apex, a distance between the first frame apex and the second frame apex while within the expanded configuration defining an expanded configuration apex distance, a distance between the first frame apex and the second frame apex while in the distended configuration defining a distended configuration apex distance; and
a support member extending between the first frame apex and the second frame apex, the support member including a first end secured to the first frame apex and a second end releasably securable relative to the second frame apex, the adjustable support adapted to limit the distended configuration apex distance relative to the expanded configuration apex distance.

14. The implantable medical device of claim 13, further comprising:
a pin secured to the second frame apex, with the support member extending through the pin; and
a tapered collar secured in position on the support member, the tapered collar adapted to be releasably secured to the pin in order to secure the second end of the support member relative to the second frame apex.

15. The implantable medical device of claim 13, wherein the support member comprises a suture that is wrapped over the first frame apex and has a first free end and a second free end that each extend proximally beyond the second frame apex.

16. The implantable medical device of claim 13, further comprising a ratcheting element, the ratcheting element including a toothed lumen through which the first free end and the second free end of the suture extends, the toothed lumen adapted to allow the ratcheting element to slide in one direction relative to the suture while restricting movement in an opposing direction.

17. The implantable medical device of claim 13, further comprising a reversible suture lock, the reversible suture lock comprising:
a suture lock body defining a lumen extending therethrough;
a first element slidingly disposed within the lumen, the first element including a first element lumen through which the suture is free to extend;
a second element slidingly disposed within the lumen and engaged with the first element, the element member including a second element lumen through which the suture is free to extend;
an engagement element that is adapted to releasably engage the first element, the engagement element including an engagement element lumen through which the suture is free to extend;
wherein the first element and the second element are moveable between a first position in which the suture is free to move relative to the suture lock body and a second position in which the suture is constrained against movement relative to the suture lock body.

18. The implantable medical device of claim 13, further comprising a suture lock comprising:
a suture lock body defining a lumen extending therethrough, the lumen accommodating the suture therethrough; and
a ratchet that engages the suture;
wherein the ratchet maintains a tensile force on the suture.

19. An LAAC (left atrial appendage closure) device, comprising:
an expandable frame defining a body of the implantable medical device, the expandable frame moveable between a collapsed configuration for delivery, an expanded configuration for deployment, and a distended configuration;
the expandable frame including a first frame apex and a second frame apex, a distance between the first frame apex and the second frame apex while within the expanded configuration defining an expanded configuration apex distance, a distance between the first frame apex and the second frame apex while in the distended configuration defining a distended configuration apex distance;
a support member extending between the first frame apex and the second frame apex, the support member including a first end secured to the first frame apex and a second end releasably securable relative to the second frame apex; and
an adjustable support adapted to limit the distended configuration apex distance relative to the expanded configuration apex distance.

20. The LAAC device of claim 19, wherein the adjustable support further comprises:
a pin secured to the second frame apex, with the member extending through the pin; and
a tapered collar secured in position on the member, the tapered collar adapted to be releasably secured to the pin in order to secure the second end of the member relative to the second frame apex.
